(19) United States
(12) Patent Application Publication
(10) Pub. No.: US 2021/0290715 A1
(43) Pub. Date: Sep. 23, 2021

(54) ANTI-INFLUENZA VIRUS COMPOSITION FOR MUCOUS MEMBRANES

(71) Applicant: Natural Healthcare Co., Ltd., Seoul (KR)

(72) Inventors: Heun Soo Kang, Seoul (KR); So Hyun Park, Gyeonggi-do (KR); Jeong Hee Hwang, Seoul (KR)

(73) Assignee: Natural Healthcare Co., Ltd., Seoul (KR)

(21) Appl. No.: 16/321,618

(22) PCT Filed: Jul. 28, 2017
(86) PCT No.: PCT/KR2017/008203

§ 371 Date: Jan. 29, 2019

(30) Foreign Application Priority Data
Jul. 29, 2016 (KR) 10-2016-0096727

(51) Int. Cl. A61K 36/258; A61K 8/9789; A61K 8/02; A61K 8/04; A61Q 17/00; C12P 1/04; A61Q 19/10; A61Q 5/02; A61Q 11/00

(52) U.S. Cl. CPC A61K 36/258; A61K 8/9789; A61K 8/0208; A61K 8/042; A61K 8/046; A61Q 17/005; C12R 2001/225; A61Q 19/10; A61Q 5/02; A61Q 11/00; A61K 2236/19; A61K 2800/85; A61K 2800/92; C12P 1/04

(57) ABSTRACT

The present invention relates to an anti-influenza virus composition for mucous membranes, comprising a fermented ginseng extract or fermented red ginseng extract as an active component.

### TECHNICAL FIELD

The present invention relates to an anti-influenza virus composition for mucous membranes, comprising a fermented ginseng or fermented red ginseng as an active component.

### BACKGROUND

Ginseng is a herbaceous perennial plant, which belongs to the Panax genus of the Araliaceae plant family according to plant taxonomy, wherein about 11 species thereof have been known on the earth. Through many pharmacological experiments, which have been conducted so far, it is known that ginseng is involved in: lowering cholesterol; inhibiting lipid peroxidation; dropping blood pressure; increasing blood flow; enlarging cerebral blood vessels; accelerating cardiac functions; performing antiarrhythmic and antithrombotic actions; inhibiting platelet aggregation; having a therapeutic effect on chronic renal failure; inhibiting cytotoxicity and cancer; performing an immunoregulatory action; enhancing memory; accelerating cerebral metabolism; performing anti-stress, anti-oxidative, anti-aging and anti-ulcer actions; inhibiting gastric secretion; raising work capacity; performing radioprotective, antidiabetic and detoxicant actions; increasing hepatocellular enzymes; treating asthma; performing anti-inflammatory and analgesic actions; treating anemia; enhancing reproductive capacity and sexual performance; lowering blood-alcohol levels; and having an activity of an antiallergic, anti-cancer drug, etc.

So far, an anti-viral activity of ginseng (or red ginseng) has been known, but nothing has been known about the anti-viral activity related to a fermented ginseng or fermented red ginseng.

Throughout the present specification, reference is made to a number of papers and patent documents, and citations thereof are marked herein. The disclosure of cited papers and patent documents are incorporated herein by reference in its entirety, and thus a level of the technical field, to which the present invention pertains, as well as a content of the present invention will be described more clearly.

### PRIOR ART REFERENCES

### Patent Documents

Korean Patent Publication No. 10-2010-0124304
Korean Patent Publication No. 10-2009-0037595
Korean Patent Publication No. 10-2014-0030360

### Non-Patent Document

J. Genseng Res 38 (2014) 40-46; J. Ginseng Res 38 (2014) 226

### DETAILED DESCRIPTION OF THE INVENTION

### Technical Problem

An objective of the present invention is to provide an anti-influenza virus composition for mucous membranes, comprising a fermented ginseng or fermented red ginseng as an active component.

Also, other objective of the present invention is to provide a pharmaceutical composition for mucous membranes for preventing or treating an influenza virus-caused disease, comprising the composition.

Further, another objective of the present invention is to provide a hygiene product, which contains the composition or is coated therewith.

Furthermore, yet another objective of the present invention is to provide an anti-influenza virus spray container for mucous membranes, which is filled with the composition.

### Technical Solution

In one aspect to achieve the objectives, the present invention provides an anti-influenza virus composition for mucous membranes, comprising a fermented ginseng or fermented red ginseng as an active component.

As used herein, “ginseng” means the ginseng, which is not treated to make a red ginseng. As the ginseng, various known ginsengs may be used, for example, including Panax ginseng, P. quinquefolius, P. notoginseng, P. japonicus, P. trifolium, P. pseudoginseng and P. vietnamensis, but not limited thereto. Preferably, the ginseng of the present invention is Panax ginseng or P. notoginseng.

Also, as used herein, “red ginseng” refers to a straw-colored or weak reddish brown ginseng, which is obtained by carefully selecting a ginseng, then steaming the ginseng with skins thereon, and then drying the resulting ginseng.

As the ginseng or red ginseng, which serves as a raw material for the fermented ginseng or red ginseng of the present invention, the one collected or cultivated in nature or the one purchased out of commercially available ones may be used, but not limited thereto.

Also, as the ginseng or red ginseng, which serves as a raw material for the fermented ginseng or fermented red ginseng, not only a raw medicinal herb but also processed goods such as powder, extracted solution, powder of extracted solution, concentrated solution, powder of concentrated solution or the like may be used.

An anti-influenza virus composition for mucous membranes according to the present invention is characterized in that the composition contains a fermented ginseng and fermented red ginseng obtained by fermenting the ginseng and red ginseng.

In the present invention, the “fermented ginseng” includes, without limitation, a substance obtained by fermenting the ginseng, and the “fermented red ginseng” includes, without limitation, a substance obtained by fermenting the red ginseng.

The fermentation of the present invention is preferably performed by means of a two-step fermentation process of lactic-acid fermentation and enzymatic fermentation.

Thus, the fermented ginseng or fermented red ginseng of the present invention may be preferably prepared by putting the ginseng or red ginseng into the two-step fermentation process of lactic-acid fermentation and enzymatic fermentation, respectively.

The lactic-acid fermentation and enzymatic fermentation may be performed simultaneously or sequentially. Preferably, as for the fermentation, the enzymatic fermentation is performed after the lactic-acid fermentation process.

Lactic acid bacteria used in the lactic-acid fermentation of the present invention include various lactic acid bacteria known in the art. For example, such bacteria may be at least one selected from the group consisting of Lactococcus, Lactobacillus, Leuconostoc, Propionibacterium, Enterococcus, Bifidobacterium, Streptococcus and Pediococcus.

Preferably, the lactic acid bacteria used in the lactic-acid fermentation of the present invention may be at least one selected from the group consisting of Lactobacillus alimentarius, Lactobacillus sakei, Lactobacillus acidophilus, Lactobacillus casei, Lactobacillus gasseri, Lactobacillus delbrueckii, Lactobacillus fermentum, Lactobacillus bulgaricus, Lactobacillus helveticus, Leuconostoc mesenteroides, Streptococcus thermophilus, Streptococcus lactis, Enterococcus faecium, Enterococcus faecalis, Bifidobacterium bifidum, Bifidobacterium infantis, Bifidobacterium brave and Bifidobacterium longum.

More preferably, the lactic acid bacteria used in the lactic-acid fermentation of the present invention may be selected from the group consisting of Lactobacillus alimentarius M-2 strain (KCTC 11054 BP) and Leuconostoc mesenteroides M-3 strain (KCTC 11055 BP). Most preferably the lactic acid bacteria used in the lactic-acid fermentation of the present invention is a combination of Lactobacillus alimentarius M-2 strain (KCTC 11054 BP) and Leuconostoc mesenteroides M-3 strain (KCTC 11055 BP).

In case of the lactic acid bacteria used in the fermentation, an inoculation may be performed with live bacteria, which have been liquid-cultured in a natural medium, as they are, or may be performed with powdered strains such as a freeze-dried form of lactic acid bacteria powder.

The inoculation with lactic acid bacteria may be performed directly into an extracted solution of ginseng or red ginseng, or may be performed into water, in which slices or powder of ginseng or red ginseng roots are immersed, but not limited thereto.

The lactic-acid fermentation of the fermented ginseng or fermented red ginseng is performed at an appropriate temperature and for an appropriate time. Preferably, the lactic-acid fermentation may be performed at 25-45° C., 27-45° C., 29-45° C., 31-45° C., 33-45° C., 33-43° C. or 33-40° C. temperatures. Also, preferably the lactic-acid fermentation may be performed for 1-20, 4-20, 4-18, 4-16, 4-14, 6-14, 8-14 or 10-14 days. More preferably, the lactic-acid fermentation may be performed at 33-40° C. temperatures and for 10-14 days.

In one specific exemplary embodiment of the present invention, the lactic-acid fermentation was performed at 25-45° C. and for 1-20 days, after performing an inoculation with about 0.5 to 2 wt % of Lactobacillus alimentarius M-2 strain (KCTC 11054 BP) and Leuconostoc mesenteroides M-3 strain (KCTC 11055 BP) into water, in which powder of ginseng or red ginseng was immersed.

An enzyme used in the enzymatic fermentation of the present invention may be at least one selected from the group consisting of pectinase, cellulase, hemicellulase, xylanase, pectolyase, pectinesterase and laminarinase, but not limited thereto.

Preferably, the enzyme may be at least one enzyme selected from the group consisting of pectinase, cellulase and hemicellulase. More preferably, the enzyme may be a combination of pectinase, cellulase and hemicellulase.

Particularly, the present invention provides a method for preparing a fermented ginseng or fermented red ginseng according to the present invention, wherein the method includes: a step of performing a lactic-acid fermentation for a ginseng or red ginseng by using Lactobacillus alimentarius M-2 strain (KCTC 11054 BP) and Leuconostoc mesenteroides M-3 strain (KCTC 11055 BP); and a step of performing an enzymatic fermentation by using pectinase, cellulase and hemicellulase.

The enzymatic fermentation is performed at an appropriate temperature and for an appropriate time. Preferably, the enzymatic fermentation may be performed at 40-60° C., 42-60° C., 44-60° C., 46-60° C., 46-58° C., 46-56° C. or 47-53° C. temperatures. Also, preferably the enzymatic fermentation may be performed for 10-90, 15-90, 20-90, 30-90, 40-90, 50-90, 60-90, 60-85, 60-80 or 60-75 hours. More preferably the lactic-acid fermentation may be performed at 47-53° C. temperatures and for 60-75 hours.

Preferably, the fermented ginseng or fermented red ginseng of the present invention may be prepared in a form of extract through an extraction process after the lactic-acid fermentation and enzymatic fermentation as above.

Particularly, the extract may be prepared by performing an extraction and filtration for the fermented ginseng or fermented red ginseng, which has finished the lactic-acid fermentation and enzymatic fermentation according to the present invention, by means of water, alcohol or a mixed solvent thereof. As the extraction solvent, water, C1-4 alcohol or a mixed solvent of water and C1-4 alcohol may be used, wherein methanol, ethanol, butanol, propanol, isopropanol and the like may be used as the C1-4 alcohol solvent.

Preferably, an ethanol extract of the fermented ginseng or an ethanol extract of the fermented red ginseng may be prepared by performing an extraction for the fermented ginseng or fermented red ginseng, which has finished the fermentation process, by means of ethanol.

Also, an extract powder may be prepared by further spray-drying the extract, or a concentrate may be prepared by performing a filtration after extraction, and then concentrating a resulting filtrate, wherein a concentrate powder may be also prepared by spray-drying the concentrate again.

Thus, the fermented ginseng of the present invention includes, without limitation, a fermented product itself, an extract thereof, an extract powder, a concentrate or a concentrate powder, which are obtained by putting a raw medicinal herb of ginseng or a powder thereof, an extract, an extract powder, a concentrate or a concentrate powder into the fermentation according to the present invention, preferably the lactic-acid fermentation and the enzymatic fermentation, and the fermented red ginseng of the present invention includes, without limitation, a fermented product itself, an extract thereof, an extract powder, a concentrate or a concentrate powder, which are obtained by putting a raw medicinal herb of red ginseng or a powder thereof, an extract, an extract powder, a concentrate or a concentrate powder into the fermentation according to the present invention, preferably the lactic-acid fermentation and the enzymatic fermentation.

Preferably, the fermented ginseng or fermented red ginseng of the present invention is contained in a composition in an amount of 0.1 to 50 w/v %.

The two-step fermentation process of the lactic-acid fermentation and enzymatic fermentation according to the present invention is a process of performing a bioconversion for ginsenoside contained in the ginseng and red ginseng.

Particularly, ginsenoside such as ginsenoside F1, ginsenoside F2, ginsenoside Rh2, protopanaxatriol (PPT), compound K or protopanaxadiol (PPD) is not detected from the ginseng or red ginseng. Surprisingly, however, if the ginseng or red ginseng is put into the lactic-acid fermentation and enzymatic fermentation, at least one ginsenoside selected from the group consisting of the ginsenoside F1, ginsenoside F2, ginsenoside Rh2, PPT, compound K and PPD is produced.

In a specific experimental example, as a result of analyzing ginsenoside components from the fermented red ginseng having undergone the lactic-acid fermentation and enzymatic fermentation according to the present invention, it might be identified that the ginsenoside F1, ginsenoside F2, ginsenoside Rh2, PPT, compound K and PPD are detected therefrom.

In the present invention, the fermented ginseng or fermented red ginseng contains ginsenoside F1 in an amount of 0.05-0.50 mg/g. Preferably, the fermented ginseng or fermented red ginseng may contain ginsenoside F1 in an amount of 0.10-0.50 mg/g, 0.15-0.50 mg/g, 0.20-0.50 mg/g, 0.20-0.45 mg/g, 0.20-0.40 mg/g, 0.20-0.35 mg/g or 0.20-0.30 mg/g.

In the present invention, the fermented ginseng or fermented red ginseng contains ginsenoside F2 in an amount of 0.20-0.80 mg/g. Preferably, the fermented ginseng or fermented red ginseng may contain ginsenoside F2 in an amount of 0.25-0.80 mg/g, 0.30-0.80 mg/g, 0.35-0.80 mg/g, 0.40-0.80 mg/g, 0.45-0.80 mg/g, 0.45-0.75 mg/g, 0.45-0.70 mg/g or 0.45-0.65 mg/g.

Also, the fermented ginseng or fermented red ginseng of the present invention contains PPT in an amount of 0.10-1.10 mg/g. Preferably, the fermented ginseng or fermented red ginseng may contain PPT in an amount of 0.20-1.10 mg/g, 0.30-1.10 mg/g, 0.40-1.10 mg/g, 0.50-1.10 mg/g, 0.60-1.10 mg/g, 0.70-1.10 mg/g, 0.70-1.05 mg/g, 0.70-1.00 mg/g or 0.70-0.95 mg/g.

Further, the fermented ginseng or fermented red ginseng of the present invention contains compound K in an amount of 1.00-8.00 mg/g. Preferably, the fermented ginseng or fermented red ginseng may contain compound K in an amount of 1.50-8.00 mg/g, 2.00-8.00 mg/g, 3.00-8.00 mg/g, 3.00-7.00 mg/g, 3.00-6.00 mg/g or 3.00-5.00 mg/g.

Furthermore, the fermented ginseng or fermented red ginseng of the present invention contains ginsenoside Rh2 in an amount of 0.10-3.00 mg/g. Preferably, the fermented ginseng or fermented red ginseng may contain ginsenoside Rh2 in an amount of 0.30-3.00 mg/g, 0.60-3.00 mg/g, 0.90-3.00 mg/g, 1.20-2.70 mg/g, 1.20-2.40 mg/g, 1.20-2.10 mg/g or 1.20-1.80 mg/g.

Moreover, the fermented ginseng or fermented red ginseng of the present invention contains PPD in an amount of 0.20-5.00 mg/g. Preferably, the fermented ginseng or fermented red ginseng may contain PPD in an amount of 0.50-5.00 mg/g, 0.80-5.00 mg/g, 1.20-5.00 mg/g, 1.50-5.00 mg/g, 1.80-5.00 mg/g, 1.80-4.70 mg/g, 1.80-4.40 mg/g, 1.80-4.10 mg/g, 1.80-3.80 mg/g, 1.80-3.50 mg/g, 1.80-3.20 mg/g or 1.80-2.90 mg/g.

The fermented ginseng or fermented red ginseng of the present invention has anti-viral activity against influenza virus.

Particularly, a composition comprising the fermented ginseng or fermented red ginseng of the present invention as an active component shows an anti-influenza virus activity, which (i) alleviates a weight loss; (ii) inhibits a virus replication in lung; (iii) reduces a production of inflammatory cytokines; and (iv) inhibits an induction of lung histopathology caused by an influenza virus infection.

An influenza virus, to which an anti-viral efficacy of the composition comprising the fermented ginseng or fermented red ginseng of the present invention as an active component may be applied, includes the influenza virus, which may infect mammals or birds, for example, birds, humans, dogs, horses, pigs, cats, etc.

Preferably, the influenza virus is an influenza virus type A.

More preferably, the influenza virus type A may be the influenza virus type A selected from the group consisting of H1N1, H5N1 and H3N2.

An anti-influenza virus composition of the present invention may further contain propolis.

Also, the propolis may be contained in the composition, for example, in an amount of 0.1 to 20 w/v %, but not limited thereto.

The anti-influenza virus composition comprising the fermented ginseng or fermented red ginseng of the present invention as an active component is characterized in that the composition is for mucous membranes.

As used herein, the “for mucous membranes” means a drug delivery into mucous membranes, that is, mucosal administration, and includes delivering a drug by bringing the drug into contact with, attaching the same onto, dispersing the same into or permeating the same into mucous membranes.

Preferably, the mucous membranes may be nasal mucous membranes, oral mucous membranes or airway mucous membranes. Particularly the mucous membranes include all the mucous membranes in the nasal cavity, mucous membranes in the oral cavity, mucous membranes of the upper respiratory tract and mucous membranes of the lower respiratory tract.

The composition of the present invention not only has an advantage of expecting an immediate effect in such a way that such composition is administered into mucous membranes and thus directly acts on a path, into which virus penetrates, but also shows a more excellent anti-influenza virus effect than being orally administered.

The anti-influenza virus composition for mucous membranes according to the present invention may be provided in a form of spray, powder, gel, ointment or drop, and preferably may be the form of spray.

In another aspect, the present invention provides a pharmaceutical composition for mucous membranes for preventing or treating an influenza virus-caused disease, comprising an anti-influenza virus composition having the fermented ginseng or fermented red ginseng as an active component.

Also, the pharmaceutical composition may further contain propolis.

The fermented ginseng, fermented red ginseng, influenza virus, mucous membranes and propolis are the same as described above.

The influenza virus-caused disease may be at least one selected from, for example, cold, flu, cough, sneeze, runny nose, muscle pain, sore throat, rhinocleisis, laryngitis, neckache, hoarseness, headache, pain in paranasal sinuses, rhinitis, pharyngitis, bronchitis, asthma, fever, dyspnea, general lethargy and chill, but not limited thereto.

The pharmaceutical composition of the present invention may be provided in a form of spray, powder, gel, ointment or drop, and preferably may be provided in the form of spray.

The pharmaceutical composition of the present invention may further contain a pharmaceutically acceptable carrier in addition to the fermented ginseng, fermented red ginseng or propolis. As the carrier, a carrier conventionally used in producing a preparation for mucosal administration may be used, and particularly there may be saline solution, buffered saline solution, dextrose, water, glycerin, isotonic aqueous buffer solution and a combination thereof. Also, in addition to the carrier above, it is possible to appropriately combine a viscosity controlling agent, preservative, isotonic agent, pH adjuster, emulsifier, inactivating agent, sweetener, perfume, acidifier, etc.

As the viscosity controlling agent, the viscosity controlling agent selected from, for example, gellan gum, xanthan gum, guar gum, sodium alginate and CMC sodium may be used.

Also, as the emulsifier, polysorbate, glycerin fatty acid ester, sodium lauryl sulfate, sorbitan fatty acid ester, sucrose fatty acid ester, polyglycerin fatty acid ester or polyoxyethylene sorbitan fatty acid ester or monoglyceride may be used.

Further, as the preservative, sodium benzoate, p-methyl benzoate, p-ethyl benzoate, p-propyl benzoate, sorbic acid, sodium sorbate or potassium sorbate may be used, and a complex Scutellaria baicalensis extract, which is a natural preservative, may be also used.

Furthermore, as the sweetener, a sugar alcohol such as D-sorbitol, D-mannitol and xylitol; a common sweetener such as sugar, glucose, maltose, fructose, etc.; a high intensity sweetener such as stevioside, enzymatically modified stevia, sucralose, aspartame, acesulfame, saccharin, etc.; polysaccharide such as dextrin, cyclodextrin, etc. may be used.

Moreover, as the perfume, a natural perfume (herbal, mint, strawberry flavor, vanilla flavor, honey flavor, etc.) or a synthetic perfume generally usable in food may be used.

In addition, as the acidifier, citric acid, acetic acid, malic acid, fruit juice, tartaric acid, formic acid, natural extract, etc. may be used.

Besides, in another aspect, the present invention provides a hygiene product, which contains an anti-influenza virus composition having the fermented ginseng or fermented red ginseng as an active component, or which is coated therewith. The anti-influenza virus composition may further contain propolis.

The fermented ginseng, fermented red ginseng, influenza virus and propolis are the same as described above.

The hygiene product of the present invention may be at least one selected from the group consisting of soap, wet tissue, tissue, shampoo, mouth freshener, air freshener and cleansing gel, but not limited thereto.

Also, in another aspect, the present invention provides an anti-influenza virus spray container for mucous membranes, which is filled with an anti-influenza virus composition for mucous membranes, comprising the fermented ginseng or fermented red ginseng as an active component. The anti-influenza virus composition may further contain propolis.

The fermented ginseng, fermented red ginseng, mucous membranes, influenza virus and propolis are the same as descried above.

The spray container of the present invention may be sprayed in an aerosol or mist phase onto mucous membranes, for example, nasal, oral or airway mucous membranes, to which the composition comprising the fermented ginseng or fermented red ginseng of the present invention is driven up and applied.

The spray container may be preferably an aerosol spray, push-to-type spray or nebulizer, but not limited thereto.

The aerosol spray may be provided by means of a device as shown exemplarily in FIG. 8. Particularly, the aerosol spray of FIG. 8 includes: a container for providing space to be filled with the composition comprising the fermented ginseng or fermented red ginseng; an operation button, which is installed in the container and manipulated in a push-to-type manner to spray the composition filling the inside of the container; and an injection nozzle for shooting the compressively stored composition in a spray form into the air. If the operation button is pressed, the compressively stored composition is shot into the air along with blowing gas by means of an internal pressure.

At this time, the blowing gas plays a role not only as a propellant for shooting the composition into the air, but also as a blowing agent for forming air bubbles in the composition for spray. As such blowing gas, at least one gas selected from liquefied natural gas (LNG), liquefied petroleum gas (LPG), butane gas, isobutane gas, propane gas and dimethyl ether (DME)-blended mixture gas may be used. The composition comprising the fermented ginseng or fermented red ginseng of the present invention as an active component may be compressively filled into the spray container by means of the blowing gas as above.

Also, the push-to-type spray may be provided by means of an oral spray device as shown exemplarily in FIG. 9. Particularly, FIG. 9 includes: a container for providing space to be filled with the composition comprising the fermented ginseng or fermented red ginseng; an operation button, which is installed in the container and manipulated in a push-to-type manner to spray the composition filling the inside of the container; and an injection nozzle for shooting the composition in a spray form into the air.

Further, the push-to-type spray may be provided by means of a nasal spray device as shown exemplarily in FIG. 10. Particularly, FIG. 10 includes: a container for providing space to be filled with the composition comprising the fermented ginseng or fermented red ginseng; an operation button, which is installed in the container and manipulated in a push-to-type manner to spray the composition filling the inside of the container; and an injection nozzle for shooting the composition in a spray form into the air.

The spray container may further include a natural extract, considering a user's taste, etc. The natural extract may be a balloon flower extract or a peppermint extract, but not limited thereto, and may be a conventional natural extract, which does not influence an effect of the fermented ginseng or fermented red ginseng on preventing and treating an influenza virus-caused disease.

Also, in another aspect, the present invention provides a method for preventing or treating an influenza virus-caused disease, wherein the method includes a step of mucosal administering the pharmaceutical composition comprising fermented ginseng or fermented red ginseng as an active component.

Besides, the pharmaceutical composition may further contain propolis.

The fermented ginseng, fermented red ginseng, influenza virus, influenza virus-caused disease, mucosal administration and propolis are the same as described above.

An appropriate dosage of the composition according to the present invention may be variously prescribed by means of factors such as a method for formulating into preparation, a patient's age, weight, gender, pathological condition, food, administration time, administration route, excretion rate and reaction sensitivity.

Preferably, the composition of the present invention may be exemplarily administered with 0.001-200 mg/kg per day in 2-20 divided doses.

Also, in another aspect, the present invention provides a use of the pharmaceutical composition for mucous membranes, comprising the fermented ginseng or fermented red ginseng as an active component, in preparing a drug for preventing or treating an influenza virus-caused disease.

Besides, the pharmaceutical composition may further contain propolis.

The fermented ginseng, fermented red ginseng, influenza virus, influenza virus-caused disease, mucosal administration and propolis are the same as described above.

Also, in another aspect, the present invention provides a veterinary drug medicine for preventing or treating an influenza virus-caused disease, comprising an anti-influenza virus composition having the fermented ginseng or fermented red ginseng as an active component.

Besides, the pharmaceutical composition may further contain propolis.

The fermented ginseng, fermented red ginseng, influenza virus, influenza virus-caused disease, mucosal administration and propolis are the same as described above.

### Advantageous Effects

A composition containing a fermented ginseng or fermented red ginseng of the present invention as an active component may be valuably utilized as an anti-influenza virus agent by alleviating a weight loss caused by an influenza virus infection; inhibiting a virus replication in lung; reducing a production of inflammatory cytokines; and inhibiting an induction of lung histopathology. Furthermore, the composition is administered into mucous membranes, for example, nasal mucous membranes, oral mucous membranes or airway mucous membranes, and thus shows a faster and more excellent anti-influenza virus activity compared to an oral administration thereof.

### BRIEF DESCRIPTION OF THE DRAWINGS

FIG. 1 shows an anti-viral protective effect of fermented red ginseng specimens A and B on H5N1 influenza virus in a mouse infection model (CD4 C57BL/6). FIG. 1 shows a change in body weights of a mouse group (n=5), which was given a mixture of the fermented red ginseng specimen A (or B) (250 μg, 1 w/v % aqueous solution) and H5N1 influenza virus. A “non-fermented red ginseng sample” means a control group, which was given a mixture of a non-fermented red ginseng extract (500 μg, 1 w/v % aqueous solution) and H5N1 influenza virus.

FIG. 2 shows a high anti-viral activity of the fermented red ginseng specimen A against H5N1 and H3N2 influenza viruses. FIG. 2 shows a change in body weights of mice (n=5, CD4 C57BL/6), which were given a mixture of influenza virus (H5N1 or H3N2) and different amounts of the fermented red ginseng (specimen A or B).

FIGS. 3A and 3B show an inhibitory effect of the fermented red ginseng specimen on a replication of H5N1 influenza virus. FIGS. 3A and 3B show a change in body weights of a mouse group (n=5, CD4 C57BL/6), which was given the mixture of H5N1 influenza virus and the fermented red ginseng specimen A or B, as well as results of analyzing a virus concentration in lung on the 6th day. “H5N1 only” means a control group, which was given the virus only without the red ginseng specimen.

FIGS. 4A to 4C show an inhibitory effect of the fermented red ginseng specimen A on a production of inflammatory cytokines caused by H5N1 influenza virus. FIGS. 4A to 4C show results of analyzing inflammatory cytokines in lung and bronchoalveolar lavage fluid (BALF) of a mouse group (n=5, CD4 C57BL/6), which was given the mixture of H5N1 influenza virus and the fermented red ginseng specimen A or B, on the 6th day. “H5N1 only” means a control group, which was given the virus only without the red ginseng specimen.

FIG. 5 shows an inhibitory effect of the fermented red ginseng specimen A on an induction of lung histopathology caused by H5N1 influenza virus. A histopathological analysis was performed by extracting a lung of a mouse group (n=5, CD4 C57BL/6), which was given the mixture of H5N1 influenza virus and the fermented red ginseng specimen A or B, on the 6th day. “Red ginseng A” and “Red ginseng B” show results of mice, which were given the mixture of the fermented red ginseng specimen A and H5N1 influenza virus, and the mixture of the fermented red ginseng specimen B and H5N1 influenza virus, respectively; “H5N1” means a control group, which was given H5N1 influenza virus only; and a “non-dosed group” is a control group, which was not given the fermented ginseng and virus.

FIGS. 6A and 6B show a protective effect of the fermented red ginseng specimen A on treatment before or after a virus infection. FIG. 6A shows a change in body weights of mice (CD5 C57BL/6) with regard to treatment with the fermented red ginseng specimen A before or after the infection with H5N1 influenza virus. FIG. 6B shows a pre-treatment effect of the fermented red ginseng specimen A on H1N1 pandemic influenza virus.

FIG. 7 shows an anti-viral effect of the fermented red ginseng specimen A on H5N1 influenza virus in an immunodeficient mouse. A μMT mouse is a mouse, which is deficient in B cells for producing antibodies. “CD4 KO 250 μg” means a CD4-deficient mouse, which was given 250 μg of the fermented red ginseng specimen A by means of 1 w/v % aqueous solution.

FIG. 8 shows a view of illustrating an example of an aerosol spray for mucous membranes according to the present invention.

FIG. 9 shows a view of illustrating an example of a push-to-type oral spray according to the present invention.

FIG. 10 shows a view of illustrating an example of a push-to-type nasal spray according to the present invention.

### MODE FOR INVENTION

Hereinafter, the present invention will be described in detail through preferred Examples for better understanding of the present invention. However, the following Examples are provided only for the purpose of illustrating the present invention, and thus the present invention is not limited thereto.

### Example 1: Preparation of a Fermented Red Ginseng

Red ginseng (Panax ginseng) provided from Ginseng Nonghyup (national agricultural cooperative federation) was ground through a 20-30 mesh, then put into a fermentation tank with an addition of purified water in an amount equivalent to 20 times more than a weight of the red ginseng, then mixed well, then sterilized by pressurizing at a high temperature of 120° C. and at 1.5 atmospheric pressure for 15 minutes. Then, a temperature of the fermentation tank was cooled down to 37° C. and kept at the same temperature, during which a sterilized mixture of red ginseng powder was inoculated with 1% (v/v) Lactobacillus alimentarius M-2 strain (KCTC11054BP) and Leuconostoc mesenteroides M-3 strain (KCTC11055BP), which are the strains of the Korean Patent Registration No. 10-0856790. After inoculation, fermentation was performed for a pre-determined period of time (12 days for the fermented red ginseng specimen A and 5 days for the fermented red ginseng specimen B), while a temperature of the fermentation tank was kept at 37° C. Then, a temperature of the fermentation tank was raised up to 50° C., then kept at the same temperature, during which a complex enzyme (Citrozym Cloudy, Novozyme) of pectinase, cellulase and hemicellulase, which are the enzymes of the Korean Patent Registration No. 10-0877489, was added 5% (v/v) thereinto, and then was subjected to reaction at 50° C. for a pre-determined time (72 hours for the fermented red ginseng specimen A and 24 hours for the fermented red ginseng specimen B). After finishing the fermentation, the mixture of red ginseng powder was sterilized in a fermentation culture tank at 95° C. for two hours, then put into an extractor with an addition of 70% ethyl alcohol by such an amount as to be five times more than the mixture, and then an extraction was performed at a temperature of 70° C. for eight hours repeatedly three times. A resulting extracted solution was filtered through a 10-50 microfilter, and a filtered extracted solution was vacuum-concentrated at a temperature of 60° C. and under a reduced pressure of 600-700 mmHg such that a solid content may reach 55%, and thus a concentrated solution of the fermented red ginseng (fermented red ginseng specimens A and B) was prepared.

### Examples 2-1 to 2-9: Preparation of a Liquid Composition for a Spray Dosage Form of the Fermented Red Ginseng

The fermented red ginseng specimen A, which was prepared in Example 1, as well as propolis and purified water were weighed to meet the contents described in a following table 1, then put into a ready preparation tank, then stirred for 20 to 60 minutes, and thus a homogenized liquid composition for a spray dosage form for mucous membranes was prepared.

TABLE 1

| | Content (in total amount of 100 mL) | | | | | | | | | | | | | | | | | |
|---|---|---|---|---|---|---|---|---|---|---|---|---|---|---|---|---|---|---|
| | Example | | Example | | Example | | Example | | Example | | Example | | Example | | Example | | Example | |
| Component | 2-1 | | 2-2 | | 2-3 | | 2-4 | | 2-5 | | 2-6 | | 2-7 | | 2-8 | | 2-9 | |
| Fermented | 0.1 | g | 5 | g | 10 | g | 30 | g | 50 | g | 5 | g | 5 | g | 5 | g | 5 | g |
| red | | | | | | | | | | | | | | | | | | |
| ginseng | | | | | | | | | | | | | | | | | | |
| Propolis | — | | — | | — | | — | | — | | 0.1 | g | 3 | g | 10 | g | 20 | g |
| Purified | Drop | | Drop | | Drop | | Drop | | Drop | | Drop | | Drop | | Drop | | Drop | |
| water | dose | | dose | | dose | | dose | | dose | | dose | | dose | | dose | | dose | |
| Total | 100 | mL | 100 | mL | 100 | mL | 100 | mL | 100 | mL | 100 | mL | 100 | mL | 100 | mL | 100 | mL |

### Examples 3-1 to 3-9: Preparation of an Aerosol Spray Container Including the Fermented Red Ginseng

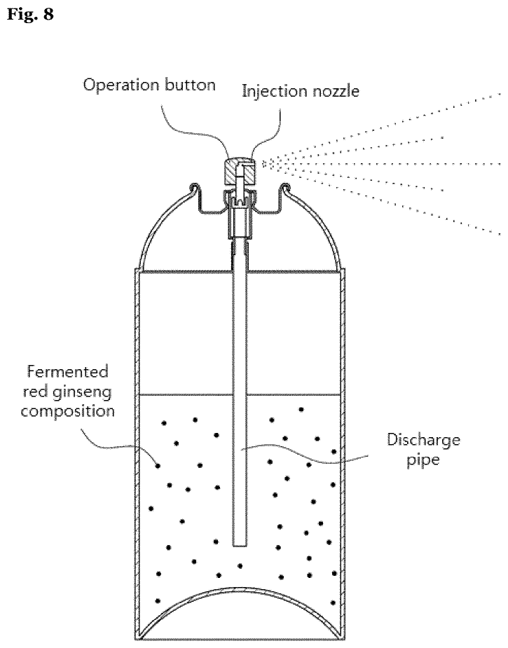

As shown in FIG. 8, a spray container including: a container capable of filling; a push-to-type operation button; and an injection nozzle was filled with a liquid composition of Examples 2-1 to 2-9, and thus an aerosol spray container including the fermented red ginseng was prepared.

### Examples 4-1 to 4-9: Preparation of a Push-to-Type Oral Spray Container Including the Fermented Red Ginseng

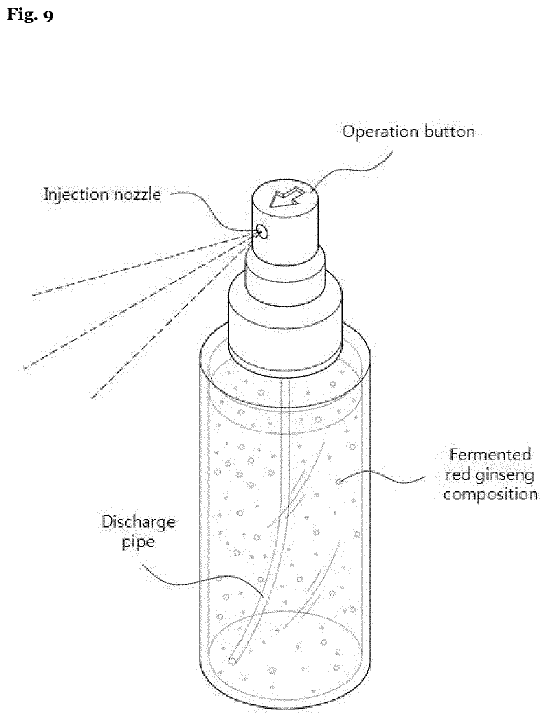

As shown in FIG. 9, a spray container including: a container capable of filling; a push-to-type operation button; and an injection nozzle was filled with the liquid composition of Examples 2-1 to 2-9, and thus a push-to-type oral spray container including the fermented red ginseng was prepared.

### Examples 5-1 to 5-9: Preparation of a Push-to-Type Nasal Spray Container Including the Fermented Red Ginseng

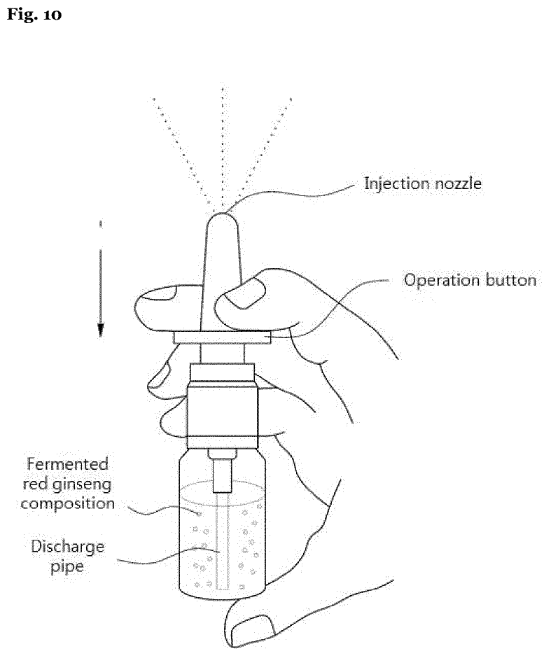

As shown in FIG. 10, a spray container including: a container capable of filling; a push-to-type operation button; and an injection nozzle was filled with the liquid composition of Examples 2-1 to 2-9, and thus a push-to-type nasal spray container including the fermented red ginseng was prepared.

### Experimental Example 1: Analysis of Ginsenoside in the Fermented Red Ginseng

Methanol was added into a vacuum-dried matter, then filtered, and then analyzed by means of HPLC. The HPLC analysis was performed by using Waters HPLC (600 controller, 717 plus Autosampler, 2487 dual absorbance detector) and a column of ZORBAX Edipse XDB-C18 (4.6×250 mm, 5 micron), wherein an oven temperature of the column was 30° C. and a loading amount of a sample was 10 μl. A mobile phase was obtained by applying a concentration gradient of 100% water and 100% acetonitrile for 75 minutes. A flow rate was 2.5 ml per minute and a detection was made at 203 nm.

As a result, it might be identified that the fermented red ginseng specimen A and fermented red ginseng specimen B contain ginsenoside F1, F2, protopanaxatriol (PPT), compound K, Rh2 and protopanaxadiol (PPD) components, as shown in a table 2. Such components of the fermented red ginseng specimens A and B are the components specific to the fermented red ginseng, which are not detected from a conventional red ginseng concentrated solution (Cheongkwanjang, Republic of Korea) (Table 2).

TABLE 2

| | Fermented | Fermented | Red ginseng |
|---|---|---|---|
| | red ginseng | red ginseng | concentrated |
| Ginsenoside | specimen A (mg/g) | specimen B (mg/g) | solution (mg/g) |
| Rg1 | 0.60 | 1.27 | 1.36 |
| Re | 0.49 | 0.87 | 1.43 |
| Rf | 0.29 | 0.13 | 1.02 |
| Rb1 | 0.95 | 2.74 | 6.86 |
| Rc | 0.04 | 0.05 | 2.76 |
| Rg2 | 0.65 | 0.25 | 1.03 |
| Rh1 | 0.58 | 0.19 | 1.02 |
| Rb2 | 0.02 | 0.26 | 2.46 |
| Rb3 | 0.01 | 0.02 | 0.57 |
| F1 | 0.28 | 0.17 | — |
| Rd | 0.62 | 0.75 | 0.82 |
| F2 | 0.55 | 0.47 | — |
| Rg3 | 0.62 | 0.54 | 1.51 |
| PPT | 0.88 | 0.33 | — |
| CK | 4.19 | 1.69 | — |
| Rh | 1.49 | 0.43 | — |
| PPD | 2.13 | 0.93 | — |
| Total | 14.39 | 11.09 | 20.84 |
| ginsenoside | | | |

### Experimental Example 2: Anti-Viral Activity of the Fermented Red Ginseng—Identification of Weight and Survival Rate

<Preparation of Virus and Administration Thereof into a Nasal Cavity of a Mouse>

As for virus, H5N1, A/Puerto Rico/8/1934 (H1N1; A/PR8) and A/Philippines/82 (H3N2 subtype) were cultured in embryonated hen's eggs by means of a known method (Quan F S et al. (2007) J Virol 81: 3514.524; Song J M et al. (2011) Proc Natl Acad Sci USA 108: 757.61; Song J M et al. (2011) PLoS One 6: e14538; Kim M C et al. (2013) Mol Ther 21: 485.92).

As for a mouse, a female BALB/c mouse (six weeks old, Harlan Laboratories) was used at a ratio of 5-6 mice per group. For an intranasal administration, the mice were anesthetized with isoflurane and given the fermented red ginseng specimen A or B, and H5N1, A/PR8 H1N1 virus (2.5 LD50) or A/Philippines/82 H3N2 virus (2.5 LD50). The mice infected with virus were observed daily and their body weights and survival rates were recorded. As for a control group, a non-fermented red ginseng extract was used.

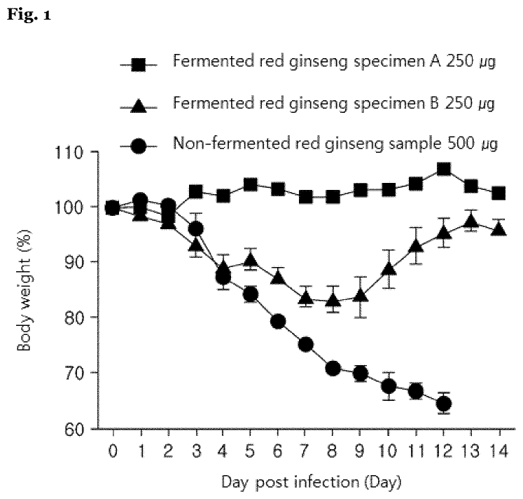

As a result, the fermented red ginseng specimens A and B showed an anti-viral effect on rgH5N1 bird flu virus, as might be identified in FIG. 1. Particularly, as a result of administering a low dose (250 μg/mouse) of the fermented red ginseng specimens A and B into a nasal cavity of each mouse by means of 1 w/v % aqueous solution, mice given a mixture of rgH5N1 virus and the fermented red ginseng specimen A did not show a weight loss, but showed a complete inhibition against a lethal dose of rgH5N1 virus. On the other hand, mice given the fermented red ginseng specimen B showed a slight weight loss, but regained a weight again in nine days later. However, mice given a non-fermented ginseng sample showed a severe weight loss and eventually died all, even if a dosage thereof doubled the fermented red ginseng specimen.

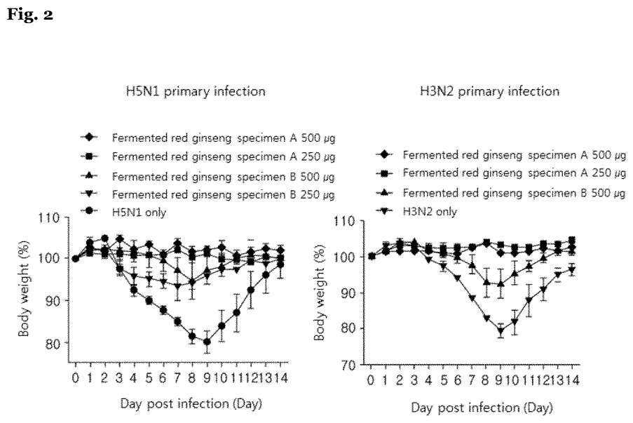

Also, a degree of anti-viral activity of the fermented red ginseng specimens A and B was identified in FIG. 2. Particularly, in mice given H5N1 and H3N2, a group of the fermented red ginseng specimen A (500 μg or 250 μg, 1 w/v % aqueous solution) did not show a weight loss, and a group of the fermented red ginseng specimen B (500 μg or 250 μg, 1 w/v % aqueous solution) showed a slight weight loss within a range of 5-15%. On the other hand, mice given H5N1 and H3N2 influenza viruses only showed a severe weight loss all.

### Experimental Example 3: Anti-Viral Activity of the Fermented Red Ginseng—Analysis of Virus Concentration

To figure out a protective efficacy of the fermented red ginseng specimens A and B on anti-viral activity, a virus concentration of mouse lungs was analyzed on the 6th day after being infected with H5N1 virus.

The virus concentration of the lungs was analyzed with MDCK cells by means of a known method (Quan F S et al., J Virol (2008) 82:1350-1359). The lungs were extracted in six days after being infected with influenza virus. Briefly, lung extracts were inoculated by serial dilution into a 6-well plate, which was seeded with MDCK cells in a single layer, and infected at 37° C. for one hour.

An overlay medium comprising DEAE dextran, non-essential amino acid, glutamine and trypsin was added thereinto, and cultured for two or three days. The cells were fixed with 0.25% glutaraldehyde, then dyed with 1% crystal violet, and then plaque was counted.

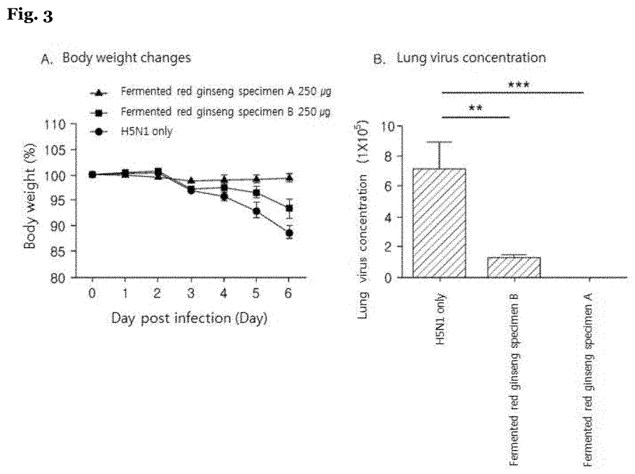

As a result, a pattern of weight changes was similarly observed in the fermented red ginseng specimens A and B (FIG. 3A). On average, 7.4×10⁵ PFU (particle forming units) of virus was detected from a lung of the mouse infected with H5N1 virus (FIG. 3B). Mice given the mixture of the fermented red ginseng specimen B and virus showed a significantly low virus concentration of 1.5×10⁵ PFU (FIG. 3B). Surprisingly, a mouse group given the fermented red ginseng specimen A showed a virus concentration in lung, which was less than or equal to a detection limit (FIG. 3B). It means that the fermented red ginseng specimen A completely inhibits a virus replication in the lung against H5N1 influenza virus.

### Experimental Example 4: Anti-Viral Activity of the Fermented Red Ginseng—Analysis of Cytokine Concentration

To figure out a protective effect of the fermented red ginseng specimen A on an inflammatory disease, a degree of pro-inflammatory cytokines in a lung extract of mice was analyzed on the 6th day after being infected with H5N1 influenza virus. A cytokine ELISA was performed by means of a known method (Quan F S et al., Vaccine (2007) 25:72-28). Cytokines were detected in the lung extract according to a manufacturer's recommended procedure by using Ready-Set-Go TNFα and IL-6 (eBioscience, San Diego, Calif.).

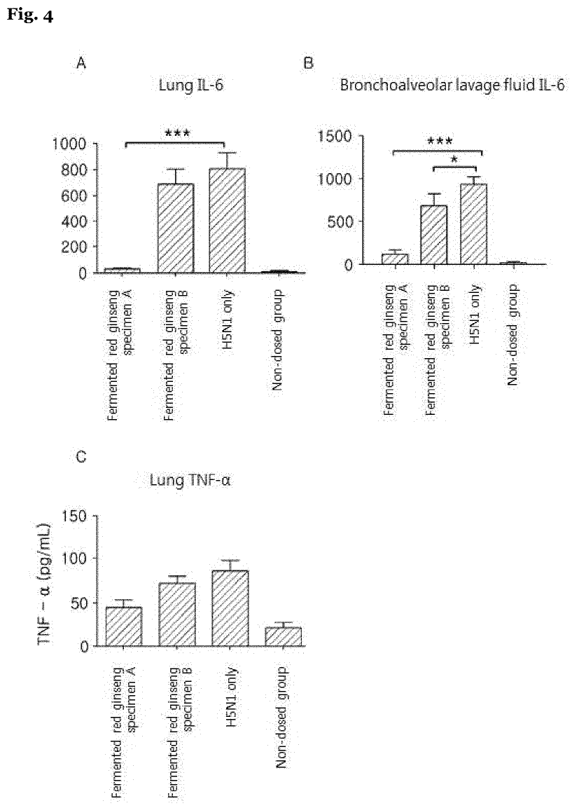

As a result, mice given H5N1 influenza virus only showed a high concentration of inflammatory cytokines (IL-6, TNF-α) in the lung and bronchoalveolar lavage fluids (BALF) (FIGS. 4A to 4C). A mouse group given the mixture of H5N1 influenza virus and the fermented red ginseng specimen B showed a low concentration of inflammatory cytokines in the BALF (FIG. 4B). Both the fermented red ginseng specimens A and B showed an effect of reducing inflammatory cytokines in the lung compared to a dosed group with H5N1 influenza virus only (FIGS. 4A and 4C). In particular, a mouse group given the mixture of the fermented red ginseng specimen A and H5N1 influenza virus showed that inflammatory cytokines were almost completely inhibited at an almost similar degree to a non-dosed mouse group (FIGS. 4A to 4C). Thus, such results mean that the fermented red ginseng specimen inhibits a production of inflammatory cytokines caused by H5N1 influenza virus.

### Experimental Example 5: Anti-Viral Activity of the Fermented Red Ginseng—Analysis of Histopathological Effect

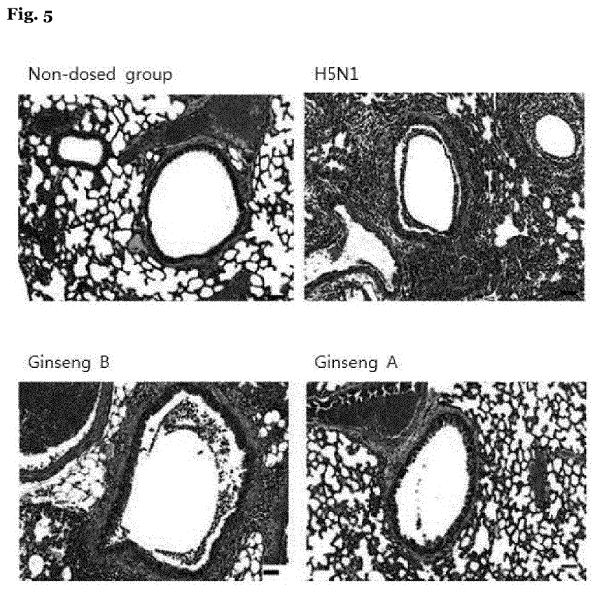

An infection with influenza virus causes severe lung inflammations along with highly invasive cells in a respiratory tract and parenchymal tissue (H5N1 of FIG. 5). A histopathological analysis was performed on a lung tissue section of each group on the 6th day after being infected with H5N1 influenza virus (FIG. 5). The mouse group given the mixture of the fermented red ginseng specimen B and H5N1 influenza virus showed that a degree of lung inflammations was alleviated more than in the mouse group given H5N1 influenza virus only. In particular, on the 6th day after being given the mixture of the fermented red ginseng specimen A and H5N1 influenza virus, the mice showed a lung histopathology at a similar degree to the lungs of non-dosed mice (FIG. 5).

### Experimental Example 6: Anti-Viral Activity According to a Nasal Mucosal Administration of the Fermented Red Ginseng

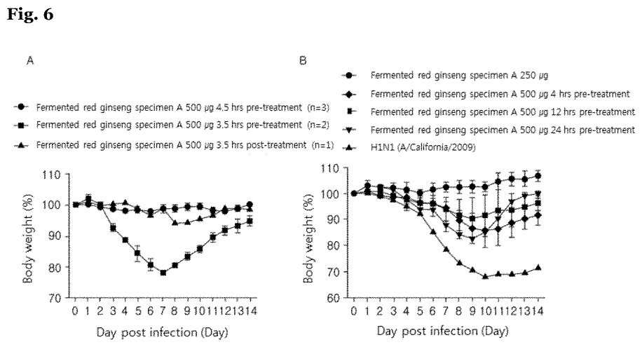

To verify preventive and therapeutic effects of the fermented red ginseng on an influenza virus infection, a protective effect thereof before and after the virus infection was determined. To test the preventive effect of the fermented red ginseng, the fermented red ginseng specimen A (500 μg) was intranasally administered into mice by means of 1 w/v % aqueous solution 4, 4.5, 12 or 24 hours before the virus infection (FIG. 6). To test the therapeutic effect of the fermented red ginseng, the fermented red ginseng specimen A (500 μg) was intranasally administered into the mice infected with influenza virus by means of 1 w/v % aqueous solution in 3.5 hours after the virus infection (FIG. 6).

As a result, it might be identified that a treatment with the fermented red ginseng has the protective and therapeutic effects in both pre-treatment and post-treatment with the H5N1 influenza virus infection. In particular, an excellent protective effect might be observed in the pre-treatment (FIG. 6A).

Also, the mice treated with the mixture of virus and the fermented red ginseng specimen showed a complete inhibition against H1N1 influenza virus (A/California/2009 pandemic virus) compared to the mice infected with virus only (FIG. 6B).

From the results, it was identified that the fermented red ginseng shows preventive and therapeutic effects on the influenza virus infection and thus may be also applied to actual treatment.

### Experimental Example 7: Effect in an Immunodeficient Mouse

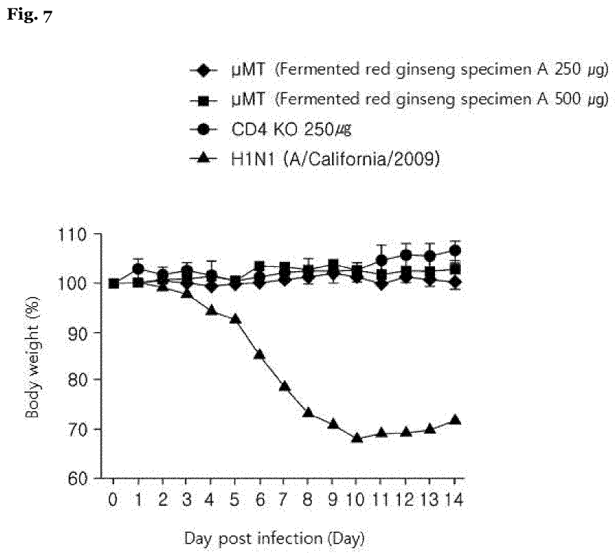

It is known that a production of antibodies is closely associated with an immunized protection against influenza virus. In other words, it means that mice deficient in B cells for producing antibodies are hardly protected from an influenza virus infection. It was investigated if the fermented red ginseng specimen A has an anti-viral protective effect on the H1N1 influenza virus infection in the B-cell-deficient mice. A μMT mouse model was used after modifying the previously mentioned protocol. As might be identified in FIG. 7, as a result of infecting the μMT mice with the mixture of the fermented red ginseng specimen A and H5N1 influenza virus (A/California/2009 pandemic virus), a complete protection was shown as observed in the other mice (CD4 C57BL/6, TLR4 C57BL/6). Such protective effect in the B-cell-deficient μMT mice is consistent with the result that the fermented red ginseng specimen may inhibit a virus replication. The fermented red ginseng specimen showed an excellent anti-viral activity even in mice without B cells for producing antibodies.

### Experimental Example 8: Evaluation of Anti-Viral Activity of a Spray Dosage Form of the Fermented Red Ginseng

To identify an anti-viral activity against H5N1 bird flu virus, the fermented red ginseng or the fermented red ginseng and propolis were administered into mice infected with H5N1 virus by using a push-to-type oral spray container of Example 4-2 or 4-7. As a result, the mice, which were given the fermented red ginseng or the fermented red ginseng and propolis after being given H5N1 virus, did not show a weight loss, but showed an inhibitory effect on a lethal dose of H5N1 virus. On the other hand, the mice, which were not given the fermented red ginseng after being given H5N1 virus, showed a severe weight loss and eventually died all.

### Experimental Example 9: Evaluation of Inhibitory Activity of the Spray Dosage Form of the Fermented Red Ginseng Against Virus Replication in the Body

To evaluate an anti-viral inhibitory efficacy of the spray dosage form of the fermented red ginseng on virus replication, the fermented red ginseng was administered into the mice infected with H5N1 virus by using a push-to-type oral spray container of Examples 4-2 and 4-7, and a virus concentration of mouse lungs was determined on the 6th day after the infection.

In case of the mice infected with H5N1 virus, a considerable amount of viruses was detected from lungs thereof. On contrary, a considerably little amount of viruses was detected from the mouse group given the fermented red ginseng or the fermented red ginseng and propolis. It means that the fermented red ginseng provided through the spray dosage form of the fermented red ginseng remarkably inhibits a virus replication in the lung against H5N1 influenza virus.

### Experimental Example 10: Histopathological Effect of the Spray Dosage Form of the Fermented Red Ginseng

An infection with influenza virus causes severe lung inflammations along with highly invasive cells in a respiratory tract and parenchymal tissue. To figure out an effect of the spray dosage form of the fermented red ginseng on lung histopathology, a histopathological analysis was performed on a lung tissue section of each group, on the 6th day after being infected with H5N1 influenza virus, by using a push-to-type oral spray container of Examples 4-2 and 4-7.

As a result, the group of mice given H5N1 influenza virus only showed considerable inflammations in the lung, but the mice showed a fair lung histopathology on the 6th day after being given the fermented red ginseng or the fermented red ginseng and propolis.

1. A method for preventing or treating an influenza virus-caused disease, wherein the method includes a step of mucosal administering a pharmaceutical composition comprising a fermented ginseng or fermented red ginseng as an active component to a subject in need thereof.

2. The method according to claim 1, wherein the fermented ginseng or fermented red ginseng is prepared by means of a two-step fermentation of lactic-acid fermentation and enzymatic fermentation.

3. The method according to claim 2, wherein the lactic acid bacteria are at least one lactic acid bacterium selected from the group consisting of Lactococcus, Lactobacillus, Leuconostoc, Propionibacterium, Enterococcus, Bifidobacterium, Streptococcus and Pediococcus.

4. The method according to claim 3, wherein the lactic acid bacteria are at least one lactic acid bacterium selected from the group consisting of Lactobacillus alimentarius, Lactobacillus sakei, Lactobacillus acidophilus, Lactobacillus casei, Lactobacillus gasseri, Lactobacillus delbrueckii, Lactobacillus fermentum, Lactobacillus bulgaricus, Lactobacillus helveticus, Leuconostoc mesenteroides, Streptococcus thermophilus, Streptococcus lactis, Enterococcus faecium, Enterococcus faecalis, Bifidobacterium bifidum, Bifidobacterium infantis, Bifidobacterium brave and Bifidobacterium longum.

5. The method according to claim 4, wherein the lactic acid bacteria are Lactobacillus alimentarius M-2 strain (KCTC 11054 BP) and Leuconostoc mesenteroides M-3 strain (KCTC 11055 BP).

6. The method according to claim 2, wherein the enzyme is at least one enzyme selected from the group consisting of pectinase, cellulase, hemicellulase, xylanase, pectolyase, pectinesterase and laminarinase.

7. The method according to claim 1, wherein the fermented ginseng or fermented red ginseng is contained in the composition in an amount of 0.1 to 50 w/v %.

8. The method according to claim 1, wherein the influenza virus is an influenza virus type A.

9. The method according to claim 8, wherein the influenza virus type A is at least one selected from the group consisting of H1N1, H5N1 and H3N2.

10. The method according to claim 1, wherein the mucous membranes are nasal mucous membranes, oral mucous membranes or airway mucous membranes.

11. The method according to claim 1, wherein the composition is in a form of spray, powder, gel, ointment or drop.

12. The method according to claim 1, wherein the subject is animal.

13. The method according to claim 1, wherein the disease is at least one selected from the group consisting of cold, flu, cough, sneeze, runny nose, muscle pain, sore throat, rhinocleisis, laryngitis, neckache, hoarseness, headache, pain in paranasal sinuses, rhinitis, pharyngitis, bronchitis, asthma, fever, dyspnea, general lethargy and chill.

14-15. (canceled)

16. A method for preparing fermented ginseng or fermented red ginseng, wherein the method includes: a step of performing a lactic-acid fermentation for a ginseng or red ginseng by using Lactobacillus alimentarius M-2 strain (KCTC 11054 BP) and Leuconostoc mesenteroides M-3 strain (KCTC 11055 BP); and a step of performing an enzymatic fermentation by using pectinase, cellulase and hemicellulase.

17. A hygiene product, which contains an anti-influenza virus composition for mucous membranes comprising fermented ginseng or fermented red ginseng as an active component, or is coated therewith.

18. The hygiene product according to claim 17, wherein the hygiene product is at least one selected from the group consisting of soap, wet tissue, tissue, shampoo, mouth freshener, air freshener, cleansing gel and anti-influenza virus spray.

19. The hygiene product according to claim 18, the hygiene product is the anti-influenza virus spray, which is filled with the anti-influenza virus composition to spray to mucous membranes.

20. The hygiene product according to claim 19, wherein the mucous membranes are nasal mucous membranes, oral mucous membranes or airway mucous membranes.

21. The hygiene product according to claim 19, wherein the spray is an aerosol spray, push-to-type spray or nebulizer.

22. (canceled)
